(19) United States
(12) Patent Application Publication
(10) Pub. No.: US 2004/0053239 A1
(43) Pub. Date: Mar. 18, 2004

(54) Dna chip for performing casual diagnosis of hypertension

(76) Inventor: Rudolf Wiesner, Frechen-Konigsdorf (DE)

Correspondence Address: Steven L Highlander, Fulbright & Jaworski, Suite 2400, 600 Congress Avenue, Austin, TX, 78701, US

(21) Appl. No.: 10/250,439

(22) PCT Filed: Dec. 28, 2001
(86) PCT No.: PCT/DE01/04960

(30) Foreign Application Priority Data
Dec. 29, 2000 (DE) 100 65 66.8

(51) Int. Cl. C12Q 1/68; C12M 1/34

(52) U.S. Cl. 435/6; 435/287.2

(57) ABSTRACT

The present invention concerns a DNA-chip for causal diagnosis of high blood pressure (hypertension), which permits early diagnosis and highly effective therapy which is tailor-made for the respective patient and which is as side-effect-free as possible. The present invention further concerns a DNA-chip for the development of new blood pressure-reducing drugs and for the stratification of patients in the early phases of clinical testing of such new drugs.

The present invention concerns a DNA-chip for causal diagnosis of high blood pressure (hypertension), which permits early diagnosis and highly effective therapy which is tailor-made for the respective patient and which is as side-effect-free as possible. The present invention further concerns a DNA-chip for the development of new blood pressure-reducing drugs and for the stratification of patients in the early phases of clinical testing of such new drugs.

About 15 million people suffer from high blood pressure in Germany, about 5 million thereof being undetected (Deutsche Bluthochdruckliga, 2000) (“German High Blood Pressure League, 2000”). High blood pressure together with the consequential illnesses thereof (arteriosclerosis, cardiac infarction, strokes, cardiac hypertrophy and cardiac insufficiency) represents by far the most frequent cause of illness and death in all Western industrial nations, even ahead of malignant degenerative illnesses (cancer). High blood pressure is due to excessive constriction of the blood vessels or inadequate excretion of fluid by the kidneys. A large number of central-nervous mechanisms and hormone systems are involved in regulation of the muscle tone of the smooth musculature in the blood vessels and thus the vessel width and also in fluid excretion by the kidneys. The mechanisms and hormone systems referred to above control and regulate the blood pressure which rises physiologically in relation to physical work, fear, stress, excitement and so forth. Derailment of one or more of those systems ultimately results in high blood pressure. In nine out of ten cases the true causes of high blood pressure are unknown (essential hypertonia). Genetic predisposition due to mutation of genes which code for proteins which are involved in blood pressure-regulating systems, sometimes only in combination with external factors (stress, smoking, overweight, lack of physical movement, poor diet) is however highly probable.

At the present time hypertension can be diagnosed only purely on a symptomatic basis, in a routine fashion by simple measurement in accordance with Riva-Rocci (arm cuff, stethoscope) or in intensive medicine in blood-invasive procedures by way of arterial catheters. This means that, in terms of the diagnosis position, the illness and possibly also negative consequential phenomena are already manifested. Diagnosis is also not without its problems in regard to limit value hypertonia as physiologically large fluctuations in blood pressure are already to be observed (for example diurnal rhythm) and also many influences can cause blood pressure to vary (for example anxiety at the examination; diagnosis; “white coat effect”). There is no early detection, that is to say a possible way of diagnosing the beginning of the illness when still at the stage of being prior to a significant rise in blood pressure. Also, in regard to a widespread disease, with such a large number of affected persons, it is completely unsatisfactory to have only a single physical method of diagnosis, that is to say to have to entirely forego pathological, clinical-chemical or apparatus options of other kinds. Also essential high blood pressure can be treated (“adjusted”) only purely symptomatically by trying out various blood pressure-reducing active substances as a monotherapy or very frequently as combination therapy as the primary causal origin in each case is unknown. A proportion of the patients however remain therapy-resistant and, even in the case of patients who are successfully treated, the findings often worsen again with increasing age. It is therefore on the one hand urgently necessary to establish an early detection method which already detects emerging hypertension, even before the physically measurable blood pressure is significantly increased, in order in good time to prevent anatomical changes to the blood vessels which are then irreversible, and further consequential damage. On the other hand it would be desirable to detect the primary cause of hypertension in each patient.

DNA-chip technology is a highly efficient method, inter alia for the detection of DNA-polymorphisms. That detection by means of DNA-chip technology is used by an already existing method of causal diagnosis of high blood pressure, that is to say, differences in the DNA-sequence of genes between various individuals are detected, which code for proteins which are responsible for blood pressure-regulating mechanisms. That method however suffers from the disadvantage that it is only in some cases that essential hypertension is monogenetic, most cases are to be attributed to a combination of mutations of which each individual one has only little predictive value. A further disadvantage is that, for ethical reasons, DNA-analysis will not gain general acceptance in regard to the predisposition for a severe disease such as high blood pressure. Nonetheless it is urgently desirable to make it clear in regard to each patient, which of the many possible, blood pressure-regulating mechanisms is disordered and is thus responsible for the individual high blood pressure of that patient. It is only diagnosis of the causes which result in high blood pressure in each individual patient, that is to say the hormone system or the central-nervous mechanism or a corresponding combination, which have suffered derailment in the individual case, that permits tailor-made therapy which is appropriately directed to the respective patient. Unwanted side-effects of unsuitable medication can also be very substantially avoided in that way. In that fashion, the successes in terms of curing this very widespread disease would be considerably improved and thus morbidity and mortality but also treatment costs would be considerably reduced.

Therefore the object of the present invention is to provide means for causal diagnosis of high blood pressure. A further object of the present invention lies in providing a method of early detection of high blood pressure. A further object of the present invention lies in providing a method of dividing hypertension patients into subclasses, for example also stratifying patients in early phases in clinical testing of new drugs.

Those objects are attained by the subject-matter defined in the claims.

The term “subclass” used here denotes a group of patients with high blood pressure, wherein the causal origin of the high blood pressure is to be attributed to a disorder of the same blood pressure-regulating system. The patients of a subclass therefore react in the same manner to a blood pressure-reducing medicament.

The term “blood pressure-regulating systems” used here denotes central-nervous mechanisms or hormonal systems which are involved in regulation of the contraction condition of the smooth musculature of the blood vessels (vessel width) or the resorption capacity of the kidney epithelium. Disordered blood pressure-regulating systems are those which are causally responsible for high blood pressure.

The term “high blood pressure” used herein denotes any kind of hypertension of stages I, II, III and IV. Depending on age hypertension usually already occurs at a systolic value above 140 mm Hg and a diastolic value of 90 to 94 mm Hg. Hypertension usually also occurs with just an increase in the diastolic value, in which respect the divisions can be as follows: mild hypertension at 95 to 104 mm Hg, mediumly severe hypertension at 105 to 114 mm Hg and severe hypertension at over 115 mm Hg.

Switching genes on and off is the basis of all biological processes and is also an extremely sensitive response to altered external conditions. DNA-chips will therefore revolutionise biomedicine and molecular biology in the forthcoming years. With the extraction of RNA from a biological sample, the action of marked cDNA or RNA itself on a DNA-chip (hybridisation) and analysis thereof, within a very short time it is possible to have a great amount of information about the gene expression profile and thus about the condition of the cells in the biological sample under altered conditions. The technology based on the hybridisation of nucleic acids has the advantages of extremely high specificity, sensitivity and being relatively easy to implement, in contrast for example to the analysis of protein patterns.

If genes are switched on and off in blood vessels or kidney tubule cells in non-physiological manner, that can be the cause of high blood pressure. The background in this respect is that all intracellular signal cascades which are involved in regulation of the vessel tone or kidney epithelium transport either directly or indirectly also influence transcription factors and thus the activity of genes. If the expression of one or more genes coding for proteins which are involved in those blood pressure-regulating systems is disturbed or if those genes carry mutations which adversely affect the function of the corresponding proteins, that has an effect on blood pressure. Switching genes on and off in blood vessels or kidney tubule cells in non-physiological manner can however also be evaluated as a measurable sign of the respectively disordered blood pressure-regulating mechanisms (surrogate marker).

An aspect of the present invention therefore lies in the provision of a DNA-chip for the causal diagnosis of high blood pressure, including at least two different nucleic acids, each coding for a respective polypeptide, wherein the polypeptides are involved in various blood pressure-regulating systems, and the nucleic acids serve as indicators for at least two disordered blood pressure-regulating systems, and optionally further including one or more nucleic acids coding for polypeptides which are expressed in each cell and which belong to the basic equipment of a cell (so-called housekeeping genes) for standardization of the signals obtained. Preferred is a DNA-chip wherein one, two, three, four, five, six or more nucleic acids of at least two blood pressure-regulating systems are contained therein. Further preferred is a DNA-chip having at least one respective nucleic acid coding for a respective polypeptide of three, four, five, six, seven, eight, nine, ten, eleven, twelve, thirteen, fourteen, fifteen, sixteen or seventeen blood pressure-regulating systems.

The nucleic acids used on the DNA-chip code for peptides which participate in biochemical processes which are involved in particular in one or more of the following blood pressure-regulating systems or however also in other blood pressure-regulating systems which are to be freshly discovered in the future:

the sympathetic-adrenergic nervous system and the adrenergic system of the suprarenal gland medulla,

the parasympathetic nervous system,

the renin-angiotensin system,

the aldosterone system,

the hypophysis-adiuretin (vasopressin) system,

the atrial-natriuretic-peptide system,

the renal kallikrein-kinin system,

the NO (nitrogen oxide) system,

the EDHF system (endothelial derived hyperpolarisation factor),

the prostaglandin system,

the endothelin system,

the PTHrP system (parathormone related peptide),

the histamine system,

the serotonin system

the purinergic system,

local growth factors, and/or

the calcium housekeeping of the smooth vessel muscle cell or the heart.

The nucleic acid used on the DNA-chip can be any nucleic acid of which it is known that it codes for a peptide which is involved in blood pressure-regulating systems or which in relation to high blood pressure is more or less expressed. In particular the nucleic acids used for the DNA-chip according to the invention are those which are selected from the following nucleic acids (the GenBank accession number is specified in each case in the brackets):

Smooth-muscular α-actin (J05192); cardial α-actin (AF053356); skeletal α-actin (AF053356); adrenergic receptor β1 (J03019); adrenergic receptor β2 (M15169); β-adrenergic receptor kinase II (M80776); adrenomedullin (D14874); angiotensin II-receptor type I (AT1) (D13814); angiotensin converting enzyme (ACE) (J04144); arginin vasopressin (AVP) (M25647); arginin vasopressin receptor (VI) (L25615); atrial natriuretic factor (M30262); brain natriuretic peptide (M25296); Ca²⁺-sarcoplasmatic reticulum ATPase (SERCA) (M63603); calcium-release channel (ryanodin receptor II) (J05200); collagen α-1 (I) (AF017178); collagen α-1 (III) (M11134); collagen α-1 (IV) (NM_{—}001845); collagen α-2 (IV) (M33653); collagen type V (BC008760); connexin 43 (Cx 43) (M65188); cytochrome P 450 CYP11-B2 (D13752); cytochrome P 450 CYP11-B1 (aldosterone synthase) (NM_{—}000497); desmin (U59167); elastin (tropoelastin) (M36860); endothelin I (ET-I) (NM_{—}001955); endothelin III (ETIII) (J05081); endothelin receptor (ETA) (L06622); endothelin receptor (ETB) (L06623); soluble E-selectin (D38257); fibroblast growth factor (bFGF) (M27968); fibronectin (X02761); G-protein, α-subunit of inhibitory (GI-α) (BC014627); G-protein-coupled receptor kinase II (L16862); guanylatcyclase, α1-subunit of the soluble (X66534); guanylatcyclase, β1-subunit of the soluble (X66533); heat shock protein 70 (hsp 70) (L12723); insulin like growth factor 1 (IGF1) (M29644); insulin like growth factor 1 receptor (NM_{—}000875); interleukin-6 (M54894); intercellular adhesion molecule (ICAM-I) (J03132); cardiotrophin 1 (CT 1) (BC012939); laminin (M20206); medium-chain acyl-coenzyme A dehydrogenase (MCAD) (M16827); mitochondrial DNA (J01415); monocyte chemoattractant protein I (MCP I) (D29984); natriuretic peptide receptor A (X15357); myosin, heavy α-chain (D00943); myosin, heavy β-chain (NM_{—}000257); myosin light chain 2 (AB046614); sodium-potassium (Na+/K+) ATPase alpha-1 subunit (U16798); sodium proton exchanger (NHE-I) (S68616); nerve growth factor (NGF) (M57399); NO-synthase endothelial (ENOS) (M95296); NO-synthase inducible (INOS) (AF051164); ornithindecarboxylase (ODC) (M16650); osteopontin (J04765); plasminogen activator inhibitor (PAI-I) (M16006); parathormone-like protein (PTH/parathyroidhormone related protein) (M17183); phosphodiesterase (L20965); phospholamban (M63603); platelet derived growth factor (PDGF-alpha) (L20965); platelet derived growth factor (PDGF-beta) (X98706); platelet derived growth factor PDGF-alpha receptor (NM_{—}006206); platelet derived growth factor PDGF-beta receptor (L20965); prostacyclin synthetase (PGI-II synthetase) (D38145); S-100 beta-protein (P04271); superoxide-dismutase (K00065); thrombospondin (NM_{—}003246); tissue plasminogen activator (tPA) (M15518); transforming growth factor β (M38449); tropomyosin, α-skeletal (M19713); troponin I, cardial (M38449); troponin I, skeletal (J04760); vascular cell adhesion molecule (VCAM-I) (X53051); vascular endothelial growth factor/permeability factor (VEGF) (AY047581); voltage-gated-K+ channel (KV1.2) (XM_{—}010737); voltage-gated-K+ channel (KV1.5) (M83254); and voltage-gated-K+ nchannel β subunit (X83127).

Particularly preferred is a DNA-chip according to the invention with the nucleic acids set out hereinafter, wherein the nucleic acids code for polypeptides which are involved in the respective specified blood pressure-regulating systems or which are differentially regulated in the event of disorders of said systems: cytochrome P 450 CYP11-B1 (aldosterone synthase) and cytochrome P 450 CYP11-B2, involved in the aldosterone system, angiotensin II-receptor type I (AT1), angiotensin converting enzyme (ACE), intercellular adhesion molecule (ICAM-I), vascular cell adhesion molecule (VCAM-I), vascular endothelial growth factor/permeability factor (VEGF), monocyte chemoattractant protein I (MCP I), plasminogen activator inhibitor (PAI-I), transforming growth factor β and fibroblast growth factor (FGF-II), involved in the renin-angiotensin system, adrenergic receptor β1, adrenergic receptor β2, β-adrenergic receptor kinase II and G-protein-coupled receptor kinase II (GRK 2), involved in the sympathetic/adrenergic system, and inducible NO-synthase (INOS), interleukin-6 and monocyte chemoattractant protein I (MCP-I), involved in the Ca²⁺-housekeeping.

In accordance with the present invention the term “proteins involved in blood pressure-regulating systems” means on the one hand that the polypeptides participate in biochemical processes in blood vessels, in the cardiac muscle or in the kidney tubule epithelium, which in the event of a malfunction result in high blood pressure. On the other hand that also means such proteins whose expression is regulated up or down by virtue of a high blood pressure (surrogate marker). Therefore in tissue samples of high blood pressure patients (for example white blood cells, blood vessel-rich tissue, cardiac muscle tissue, kidney tissue), genes can also be altered in respect of their expression, the products of which do not causally produce the high blood pressure. Such genes can also be indicative in respect of the blood pressure-regulatory systems which are disturbed in the respective patient.

The nucleic acids possibly used for standardization of the signals on the DNA-chip according to the invention can be those in respect of which it is known that they are expressed in every cell and belong to the basic equipment of the cells. Usually the genes which code for those nucleic acids are also referred to as housekeeping genes. A selection by way of example in respect of such nucleic acids is set out hereinafter: cellular β-actin, cyclophilin, glycerine aldehyde phosphate dehydrogenase (GAPDH), glycerol-3-phosphate dehydrogenase (GPDH), histones, phospholipase, ribosomal proteins, tubulin, and ubiquitin. If a blood vessel-rich tissue is used for the diagnosis procedure, smooth-muscular housekeeping genes such as for example caldesmon or calponin can be used for standardization of the signals.

The nucleic acid sequences of the nucleic acids used on the DNA-chip can be taken from the generally accessible databases (for example GenBank, EMBL), and can be picked out in accordance with the usual criteria. Preferred in that respect are those sequences which are unique, which do not have any GC-rich regions, which do not permit intramolecular hybridisation and which do not form loops. Preferably a single-strand nucleic acid is applied to the DNA-chip according to the invention. In that respect both the sense and also the antisense strand can be used. It is however also possible for a double-strand nucleic acid to be applied to the DNA-chip according to the invention. In that respect the length of the nucleic acids is not limited upwardly or downwardly as long as the length is sufficient for specific hybridisation. The nucleic acids can also be DNA-, RNA- or hybrid-molecules. The production of such nucleic acids is state of the art and it is possible for oligonucleotides, PCR products, cloned DNA- or cDNA-fragments or cRNAs after in-vitro transcription (natural or modified) to be applied and immobilized on a carrier with standard techniques. In the case of single-strand nucleic acids preferably the sequence which is complimentary to the cDNA (when using cDNA from the biological sample) or the sequence which is complimentary to the RNA (when using RNA from the biological sample) is applied.

The carrier to which the nucleic acids are applied can be any carrier which is usually employed for DNA-arrays or DNA-chips. The methods of applying and immobilizing the nucleic acids are also state of the art and known to the man skilled in the art.

The DNA-chip according to the invention can be used for example for sensitive early detection of developing hypertension, at a time which is still before high blood pressure clinically manifesting itself. The DNA-chip can also be used for dividing up the patients into subgroups. On the basis of the genes present on the DNA-chip it is possible to detect the blood pressure-regulatory systems which are disordered in the respective high blood pressure patients. The patient subgroups can then be subjected to therapy with individually suitable active substances or active substance combinations which not only eliminate the symptom of high blood pressure but which also counteract the causal origin of the high blood pressure. The DNA-chip according to the invention can also be used for the development of new blood pressure-reducing drugs or for the stratification of patients in early phases of clinical study of such new drugs. An aspect of the present invention therefore concerns the use of the DNA-chip according to the invention for the diagnosis of high blood pressure and also for early diagnosis, for dividing high blood pressure patients into subgroups with different high blood pressure-triggering causes and for monitoring high blood pressure therapy.

For that purpose blood or tissue samples are taken from the patients to be investigated. RNA can be isolated from those patient samples using standard procedures and further used either as overall RNA or after fractionating as polyA⁺-RNA. The RNA can be transcribed by means of reverse transcriptase into cDNA and in that procedure provided with a marking, for example with a fluorescing group or an isotope etc. Also, starting from the cDNA, it is possible firstly to produce and mark PCR-products which are then hybridised with the DNA-chip. In addition the RNA can be used directly marked or unmarked for hybridisation on the DNA-chip. After hybridisation of the sample nucleic acids with the target nucleic acids on the carrier, subsequent washing steps etc., the binding of sample nucleic acids to the target nucleic acids on the carrier is analysed. That can be implemented by optical processes in the case of fluorescence marking, by autoradiography or by any other process which is capable of detecting successful hybridisation of sample nucleic acids with target nucleic acids.

The tissue taken from the patients can be taken by way of example as a biopsy, for example from the blood vessel-rich mouth-nose region or from any other tissue which is blood vessel-rich but which can be taken with a relatively low level of risk, or from the cardiac muscle, for example in the context of cardiac catheterisation for determining contractility and heart capacity. In addition lymphocytes (white blood cells) can be taken from the patient from the blood.

In accordance with the invention the patients can be divided into subgroups on the basis of their gene expression profile (gene activity: ON-OFF; high-low), that is to say into subgroups in which one or more of the above-described hormonal or central-nervous signal paths (blood pressure-regulating systems) are disturbed.

By way of example in patients suffering from high blood pressure with hyperaldosteronism (aldosterone system) the genes for cytochrome P 450 CYP11-B1 (aldosterone synthase) and for cytochrome P 450 CYP11-B2 in white blood cells are activated. The patients should be treated with an aldosterone-antagonist, for example with spirolactone.

By way of example in high blood pressure patients with increased circulating angiotensin II (increased plasma renin activity; renin-angiotensin system) on the one hand the expression of the angiotensin II receptor (type 1, AT1) in white blood cells, and on the other hand the expression of the angiotensin converting enzyme, the intercellular adhesion molecule (ICAM-I), the vascular adhesion molecule (VCAM-I), the vascular endothelial growth factor/permeability factor (VEGF), the monocyte chemoattractant protein (MCP I), the PAI-1, the transforming growth factor β and the fibroblast growth factor (FGF-II) in blood vessels or the expression of the brain natriuretic peptide (PNP) in the cardiac muscle are increased. Those patients should be treated with an angiotensin II-receptor-antagonist, for example with irbesartan, or an angiotensin converting enzyme (ACE)-inhibitor, for example with enalapril or captopril.

By way of example in high blood pressure patients with stimulated sympathetic-adrenergic system (sympathetic-adrenergic nervous system and the adrenergic system of the suprarenal gland mark) on the one hand the expression of the G-protein-coupled receptor kinase II in white blood cells and on the other hand the expression of the adrenergic receptors (β1 and β2) and the β-adrenergic receptor kinase in blood vessels or the expression of ornithine decarboxylase (ODC) in the cardiac muscle are increased. Those patients are to be treated with a β-adrenergic receptor blocker, for example with atenolol or bisoprolol.

For example in high blood pressure patients with disturbed Ca⁺⁺-housekeeping of the vessel muscle cells (calcium housekeeping of the smooth vessel muscle or the heart) the expression of interleukin-6 in blood vessels is reduced. The patients should be treated with a Ca⁺⁺channel blocker, for example with verapamil, nifedipine or amlodipine.

For example in high blood pressure patients with disturbed NO-system the expression in blood vessels on the one hand of the endothelial NO-synthase (eNOS) is reduced while on the other hand that of angiotensin converting enzyme (ACE) or collagen type I is increased. These patients should be treated with NO-donors, for example with nitrates.

The following example serve only to illustrate the invention and do not in any way limit the scope of the invention.

Taking a Submucosa Biopsy from the Mouth or the Lower Nostril (Extremely Blood Vessel-Rich Tissue), from the Heart or Obtaining White Blood Cells

Both the lower lip and also the lower nostril are suitable for taking biopsies as they are highly accessible, spraying on a local anaesthetic is suitable for anaesthesia, the tissue is very rich in arterioles and venoles, but does not have any relatively large vessels or nerves, and sample-removal defects are not visible and heal quickly.

1. Lower Lip

In the case of 8 patients after the application of 1% Xylocaine spray with epinephrine addition (Xylocaine 1%®, Astra Chemie) the lower lip was lifted and a surface mucous membrane cut of 2-4 mm in length was made at the level of the gum with a scalpel size 15. Using the Grünwald cutting tweezer (Storz) a respective tissue fragment of about 250 mg in weight was submucously taken through the cut and shock-frozen in liquid nitrogen. In general further care for the sample-removal defect was not required, re-epithelisation was concluded after between 2 and 3 days. In occasional cases more severe bleeding occurred perioperatively, so that the defect was closed with a single button suture with resorbable suture material (Vicryl 3/0, Ethicon).

2. Lower Nostril

In the case of 10 patients the nasal mucous membranes were firstly sprayed with Pantocaine/Privine (1/1, Pantoprivine 2%®, Astra Chemie). After surface anaesthesia had been implemented the Grünwald cutting tweezer (Storz) was used to take a respective tissue fragment measuring about 250 mg submucously from the lateral component of the head of the concha inferior. The tissue was immediately shock-frozen in liquid nitrogen. Bipolar coagulation was effected in the event of relatively severe bleeding. A suture was not required. The defect healed in 3-4 days.

3. Cardiac Muscle Biopsy

In the case of three patients, a biopsy of about 500 mg was taken from the free chamber wall with a stamp in the context of a left-heart catheterisation operation for diagnosis of the left-ventricular function. The tissue was immediately shock-frozen in liquid nitrogen.

4. White Blood Cells:

Taking 15 patients 100 ml of blood was taken and subfractionated in conventional manner into erythrocytes, thrombocytes and lymphocytes (Lymphoprep, Nycomed, Pharma AS or by density gradient centrifuging on a ficoll/metrizoate gradient). The lymphocyte fraction was immediately subjected to further processing.

5. RNA Production:

The overall RNA was obtained from the tissue samples or the white blood cells respectively using various extraction processes by means of commercially available kits. The yield was about 0.5 mg of RNA per g of cardiac muscle, 0.1 mg of RNA per g of submucosa tissue and about 0.1 mg of RNA per lymphocyte preparation. The RNA was investigated in respect of the expression of the genes involved using conventional methods (blots, hybridisation with radioactively or non-radioactively marked probes).

6. Gene Expression Analysis by Means of DNA-Array:

RNA from tissue samples of 5 hypertension patients and a reference RNA sample, pooled from the individual RNA samples from the tissue of 5 healthy experimentees was reversely transcribed into cDNA by means of oligo-dT primers in the presence of Cy5- and Cy3-dUTP respectively (Amersham) and fluorescence-marked. The cDNA samples (experimentee and reference respectively) were each mixed in the same ratio and hybridised with oligonucleotide arrays or cDNA arrays, they were washed after hybridisation and the fluorescence signals evaluated with a DNA array reader.

1. A device comprising a carrier to which at least two different nucleic acids, each coding for a respective polypeptide, are applied, wherein the polypeptides are involved in various blood pressure-regulating systems, and the nucleic acids serve as indicators for at least two disturbed blood pressure-regulating systems, and optionally further including one or more nucleic acids, coding for polypeptides which are expressed in each cell and which belong to the basic equipment of a cell.

2. A device according to claim 1, wherein the blood pressure-regulating systems are selected from the sympathetic-adrenergic nervous system and the adrenergic system of the suprarenal gland medulla, the parasympathetic nervous system, the renin-angiotensin system, the aldosterone system, the hypophysis-adiuretin (vasopressin) system, the atrial-natriuretic-peptide system, the renal kallikrein-kinin system, the NO (nitrogen oxide) system, the EDHF system (endothelial derived hyperpolarisation factor), the prostaglandin system, the endothelin system, the PTHrP system (parathormone related peptide), the histamine system, the serotonin system, the purinergic system, local growth factors, and the calcium balance of the smooth vessel muscle cell or the heart.

3. A device according to claim 1 or claim 2, wherein the nucleic acids are selected from smooth-muscular α-actin (J05192); cardial α-actin (AF053356); skeletal α-actin (AF053356); adrenergic receptor β1 (J03019); adrenergic receptor β2 (M15169); β-adrenergic receptor kinase II (M80776); adrenomedullin (D14874); angiotensin II-receptor type I (AT1) (D13814); angiotensin converting enzyme (ACE) (J04144); arginin vasopressin (AVP) (M25647); arginin vasopressin receptor (VI) (L25615); atrial natriuretic factor (M30262); brain natriuretic peptide (M25296); Ca²⁺-sarcoplasmatic reticulum ATPase (SERCA) (M63603); calcium-release channel (ryanodin receptor II) (J05200); collagen α-1 (I) (AF017178); collagen α-1 (III) (M11134); collagen α-1 (IV) (NM_{—}001845); collagen α-2 (IV) (M33653); collagen type V (BC008760); connexin 43 (Cx 43) (M65188); cytochrome P 450 CYP11-B2 (D13752); cytochrome P 450 CYP11-B1 (aldosterone synthase) (NM_{—}000497); desmin (U59167); elastin (tropoelastin) (M36860); endothelin I (ET-I) (NM_{—}001955); endothelin III (ETIII) (J05081); endothelin receptor (ETA) (L06622); endothelin receptor (ETB) (L06623); soluble E-selectin (D38257); fibroblast growth factor (bFGF) (M27968); fibronectin (X02761); α-subunit of inhibitory G-protein (GI-α) (BC014627); G-protein-coupled receptor kinase II (L16862); α1-subunit of the soluble guanylatcyclase (X66534); β1-subunit of the soluble guanylatcyclase (X66533); heat shock protein 70 (hsp 70) (L12723); insulin like growth factor 1 (IGF1) (M29644); insulin like growth factor 1 receptor (NM_{—}000875); interleukin-6 (M54894); intercellular adhesion molecule (ICAM-I) (J03132); cardiotrophin 1 (CT 1) (BC012939); laminin (M20206); medium-chain acyl-coenzyme A dehydrogenase (MCAD) (M16827); mitochondrial DNA (J01415); monocyte chemoattractant protein I (MCP I) (D29984); natriuretic peptide receptor A (X15357); myosin, heavy α-chain (D00943); myosin, heavy β-chain (NM_{—}000257); myosin light chain 2 (AB046614); sodium-potassium (Na+/K+) ATPase alpha-1 subunit (U16798); sodium proton exchanger (NHE-I) (S68616); nerve growth factor (NGF) (M57399); endothelial NO-synthase (ENOS) (M95296); inducible NO-synthase (INOS) (AF051164); ornithindecarboxylase (ODC) (M16650); osteopontin (J04765); plasminogen activator inhibitor (PAI-I) (M16006); parathormone-like protein (PTH/parathyroidhormone related protein) (M17183); phosphodiesterase (L20965); phospholamban (M63603); platelet derived growth factor (PDGF-alpha) (L20965); platelet derived growth factor (PDGF-beta) (X98706); platelet derived growth factor PDGF-alpha receptor (NM_{—}006206); platelet derived growth factor PDGF-beta receptor (L20965); prostacyclin synthetase (PGI-II synthetase) (D38145); S-100 beta-protein (P04271); superoxide-dismutase (K00065); thrombospondin (NM_{—}003246); tissue plasminogen activator (tPA) (M15518); transforming growth factor β (M38449); tropomyosin, α-skeletal (M19713); troponin I, cardial (M38449); troponin I, skeletal (J04760); vascular cell adhesion molecule (VCAM-I) (X53051); vascular endothelial growth factor/permeability factor (VEGF) (AY047581); voltage-gated-K+ channel (KV1.2) (XM_{—}010737); voltage-gated-K+ channel (KV1.5) (M83254); and voltage-gated-K+ channel β subunit (X83127).

4. Use of the device according to one of claims 1 to 3 for the development of new blood pressure-reducing drugs, for the stratification of patients in early phases of the clinical testing of such new drugs, for the causal diagnosis of high blood pressure, for the early diagnosis of high blood pressure, for dividing high blood pressure patients into subgroups with differing high blood pressure-triggering causes or for monitoring high blood pressure therapy.
